(19) United States
(12) Patent Application Publication
(10) Pub. No.: US 2014/0318540 A1
(43) Pub. Date: Oct. 30, 2014

(54) DEVICE FOR REMOVAL OF GAS FROM A RESPIRATORY CIRCUIT

(71) Applicant: Drüger Medical GmbH, Lübeck (DE)

(72) Inventors: Andreas Brandt, Lubeck (DE); Ralf Heesch, Lubeck (DE); Gerd Peter, Lubeck (DE); Mike Dahncke, Hamburg (DE)

(21) Appl. No.: 14/357,637

(22) PCT Filed: Nov. 12, 2012
(86) PCT No.: PCT/EP2012/072370

§ 371 Date: May 12, 2014

(30) Foreign Application Priority Data
Nov. 12, 2011 (DE) 10 2011 118 435.6

(51) Int. Cl. A61M 16/00; A61M 16/08

(52) U.S. Cl. CPC A61M 16/009; A61M 16/0057; A61M 16/0883; A61M 16/0816
USPC 128/204.18

(57) ABSTRACT

A respiratory circuit gas removal device (1) has a inhalation branch subsection (2), a exhalation branch subsection (3) and a tube connection subsection (4), with a tube connection opening (5) for a tube adapter (17). The inhalation subsection connects to the tube subsection via a first opening (6), and the exhalation subsection opens into the tube subsection via a second opening (7). A gas removal tube (8) extends from the tube subsection to a removal opening (11), essentially concentric with a tube adapter opening (18). A tube end face (25) connects an edge (24) of a first end (12), which delimits an outer wall (8a) of the gas removal tube, to an inner wall (8b) of the gas removal tube surrounding the removal opening (11). At least 40 percent of the end face (25) is in a funnel shape and has an opening angle (15) between 130° and 170°.

### CROSS REFERENCE TO RELATED APPLICATIONS

This application is a United States National Phase Application of International Application PCT/EP2012/072370 filed Nov. 12, 2012 and claims the benefit of priority under 35 U.S.C. §119 of German Patent Application DE 10 2011 118 425.6 filed Nov. 12, 2011, the entire contents of which are incorporated herein by reference.

### FIELD OF THE INVENTION

The present invention pertains to a device for removal of gas from a respiratory circuit.

### BACKGROUND OF THE INVENTION

Respirator systems are usually used in medical applications for assisting breathing and/or for administering anesthetics during surgical procedures. While the respiratory gas circulates in a closed circuit and is fed to the patient again in anesthesia systems after the carbon dioxide contained in the exhaled air has been removed, an open circuit is usually used in respirator systems in intensive care, i.e., the exhaled air is fed into the room air.

In case of intubated patients, the tube is connected to the respirator system by means of a tube adapter, which is formed on the tube and which is inserted into an opening of a connection piece. The connection piece is usually of a Y-shaped design with bent-off legs, in which an opening each is provided for connecting the tube adapter as well as the inhalation branch and exhalation branch of the respirator system. Based on its shape, such a connection piece is called Y-piece.

To remove a gas sample of a respiratory gas flowing in the respirator system, a gas removal tube may be passed through the wall of the Y-piece in the Y-piece. Such a gas removal tube is used especially to remove a gas sample of the exhaled breathing gas, i.e., of the gas exhaled by a patient, in order to determine, for example, the carbon dioxide content (EtCO2) of that gas.

A Y-piece with a gas removal tube is known, for example, from U.S. Pat. No. 5,213,096 A. The gas removal tube in this case passes through the Y-piece up to the end of a connecting branch, to which a tube can be connected via a patient connection piece having a bellows-like design. The patient connection piece forms an additional volume in this case, which may have a disadvantageous effect during the respiration of newborns, because the tidal volume of newborns is only between 5 mL and 10 mL.

A Y-piece that makes possible the direct insertion of a tube adapter formed on a tube and thus has a smaller dead space is known from EP 2 359 889 A1. A gas removal tube protrudes only partially, in this case, into a connection piece used to insert the tube adapter, so that the tube adapter can be inserted. Webs, which ensure that the opening in the tube adapter cannot be covered by the gas removal tube or cannot be closed completely in the extreme case during insertion, are formed at the end of the gas removal tube pointing towards the tube adapter.

Mixing of inhaled breathing gas and exhaled breathing gas may occur in the Y-piece. Mixing with inhaled breathing gas rich in oxygen should be avoided to the greatest extent possible especially during the measurement of the CO₂ content in the exhaled breathing gas. A reduction of an undesired mixing of inhaled breathing gas and exhaled breathing gas is achieved in the Y-piece according to EP 2 359 889 A1 by the removal of gas taking place directly above the opening in the tube adapter. A weaker flow of inhaled breathing gas maintained during the phase of exhalation therefore flows predominantly above the gas removal tube, so that more accurate determination of the EtCO2 value is made possible.

### SUMMARY OF THE INVENTION

The basic object of the present invention is to provide an advantageous device for removing gas from a respiratory circuit, in which mixing of inhaled breathing gas and exhaled breathing gas is reduced further.

The device according to the present invention for removing gas from a respiratory circuit comprises a first subsection for connection to an inhalation branch of a respirator system, a second subsection for connection to an exhalation branch of the respirator system, and a third subsection with a tube connection opening for inserting a tube adapter, wherein the first subsection opens into the third subsection via a first opening and the second subsection opens into the third subsection via a second opening. The device according to the present invention comprises, furthermore, a gas removal tube, which protrudes, starting from an opening in a wall of the third subsection, into the third subsection with a first end having a removal opening, so that the removal opening is arranged essentially concentrically to an opening formed in the tube adapter with the tube adapter inserted and the first end is arranged under an edge of the first opening and an edge of the second opening relative to a direction in which the exhaled breathing gas predominantly leaves the opening in the tube adapter during the use of the device. The first end has an end face, which connects an edge of the first end, which said edge defines an outer wall of the gas removal wall, with an inner wall of the gas removal tube surrounding the removal opening. According to the present invention, at least 40% of the end face is funnel-shaped and is formed with an opening angle between 130° and 170°.

The funnel-shaped area of the end face of the first end causes at least part of the exhaled breathing gas flowing from an opening in the tube adapter with the tube adapter connected, which breathing gas flows towards the funnel-shaped area of the end face, to be deflected back in the direction of the tube and to be swirled in the process. Swirls develop within the third subsection in an area beneath the openings of the first subsection and of the second subsection and form in this manner, especially in an area located between the opening in the tube adapter and the removal opening, a barrier (a swirl barrier) for inhaled breathing gas, which flows during a phase of exhalation of a respiration cycle from the first subsection to the second subsection. Mixing of the inhaled breathing gas with exhaled breathing gas exhaled by a patient is reduced by the swirls of the exhaled breathing gas especially in the area of the removal opening. As a result, in the ideal case unmixed exhaled breathing gas reaches the removal opening from the opening in the tube adapter.

It is thus made possible by the device according to the present invention to operate a respirator system such that inhaled breathing gas can flow into the exhalation branch through the Y-piece even during the phase of exhalation via the inhalation branch, while exhaled breathing gas can be removed at the same time from the Y-piece and measurement of the CO₂ concentration can be carried out in the exhaled breathing gas with high accuracy.

A funnel-shaped design of an area of the end face is defined in the sense of the present application such that the funnel-shaped area of the end face connects the edge of the first end with the inner wall of the gas removal tube, which said inner wall surrounds the removal opening, while the first end tapers in the direction of the exhaled breathing gas flow being removed from the diameter at the edge of the first end to the diameter of the removal opening. It is irrelevant whether the tapering takes place linearly or along a bent curve. A design with a discontinuous course of the tapering, for example, through an essentially rectangular step, shall also be covered by the definition of funnel-shaped.

An opening angle is defined as the angle that is present in a section through the center of the removal opening between the straight lines that are obtained when connecting the two points that define the diameter at the edge of the first end with the respective nearest of the two points on the inner wall of the gas removal tube, which points define the diameter of the removal opening. In case of a linear taper, these straight lines are thus located on the surface. The gas removal opening may be designed, in particular, as a hollow cylinder. However, a gas removal tube that has an elliptical, rectangular or polygonal as well irregularly shaped outer and/or inner cross section over the entire length or partial areas of the gas removal tube shall also be covered.

An essentially concentric arrangement of the removal opening with the opening formed in the tube adapter shall be defined such that a deviation from an exactly concentric arrangement based on manufacturing tolerances and preferably also a structural deviation, which affects the effect of swirling only insignificantly at best, shall also be covered in any case. A deviation based on manufacturing tolerances preferably comprises a deviation by up to 20% of the diameter of the removal opening and a structural deviation comprises up to 50% of the diameter of the removal opening.

The bisecting line of the opening angle is essentially parallel to the direction in which most of the exhaled breathing gas is discharged from the opening of the connected tube adapter into the third subsection during the use of the device in a preferred embodiment of the present invention.

While the gas removal tube may also extend, for example, obliquely to the direction of the exhaled breathing gas leaving the tube adapter above the first end, the most uniform possible admission of flow to the funnel-shaped area is brought about for the exhaled breathing gas flowing from the connected tube adapter regardless of the further course of the gas removal tube due to the advantageous orientation of the funnel-shaped area of the end face of the first end, so that the most uniform swirling possible can be achieved.

The ratio of the diameter at the edge of the first end to the diameter of the tube connection opening is advantageously between 0.35 and 0.65.

It is advantageous, furthermore, if the ratio of the diameter of the removal opening to the diameter at the edge of the first end is between 0.125 and 0.4.

Especially effective swirling can be achieved due to the preferred selection of the dimensions of the first end as well as of the removal opening. The selection of the diameter at the edge of the first end and the selection of the diameter of the removal opening already have, viewed on their own, an advantageous effect on the formation of the swirl. However, a range of parameters that also satisfies the above-mentioned ratio to the diameter of the tube connection opening is especially advantageous.

Provisions are made in a variant of the present invention for at least one web to be formed at the first end of the gas removal tube as a stop. The web protrudes farther into the third subsection than the first end, so that a tube adapter can be inserted into the third subsection at most up to the web.

The tube adapter is prevented in this manner from being pressed against the first end, as a result of which the opening in the tube adapter could be closed in the extreme case. It is thus guaranteed by the at least one web that exhaled breathing gas can always flow radially to the first end in the direction of an inner wall of the third subsection and especially that swirls can form in this area. Various embodiments of such webs at one end of a gas removal tube are known to the person skilled in the art from EP 2 359 889 A1. In the embodiment with webs, the webs cover part of the end face of the first end, which end face is available without webs for the funnel-shaped area. The funnel-shaped area of the end face comprises in this case at most the area of the end face not covered by webs. If the percentage of the end face covered by the funnel-shaped area is specified in this application, this area always refers to the end face available without webs.

The part of the gas removal tube extending in the device is advantageously connected by a partition with an inner wall of the third subsection, which inner wall is arranged between the first opening and the second opening, such that when the device is used, inhaled breathing gas flowing from the first opening towards one side of the partition must flow through around the gas removal tube and/or under the partition in order to reach the other side of the partition and to be able to flow out from there via the second opening.

A flow of inhaled breathing gas, which is maintained during the phase of exhalation and flows from the inhalation branch connected to the first opening to the exhalation branch connected to the second opening will have in this manner a preferred direction around the gas removal tube, as a result of which the displacement effect of the swirl is supported.

The web and the partition are advantageously of a one-piece design.

It is especially advantageous if the first subsection extends essentially at right angles to a plane defined by the first subsection and the second subsection.

The direction of flow of the inhaled breathing gas from the first subsection into the third subsection is essentially at right angles to the direction of flow of the exhaled breathing gas flowing from the opening in the tube adapter into the third subsection in this manner. As a result, the swirls can form more easily around the gas removal tube, and a lower intensity of the swirls is necessary for displacing most of the inhaled breathing gas arriving from the first subsection from the area located under the first opening and the second opening. In particular, the displacement of the inhaled breathing gas from the area located between the opening in the tube adapter and the removal opening is thus facilitated.

The present invention also pertains to a respirator system with a device according to the present invention.

The present invention will be explained in more detail below on the basis of exemplary embodiments shown in the drawings. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a bottom view showing a device according to the present invention in the form of a Y-piece;

FIG. 2 is a sectional view along A-A through the device according to FIG. 1; and

FIG. 3 is a sectional view along B-B through the device according to FIG. 1.

### DESCRIPTION OF THE PREFERRED EMBODIMENTS

Identical reference numbers in the figures designate identical objects.

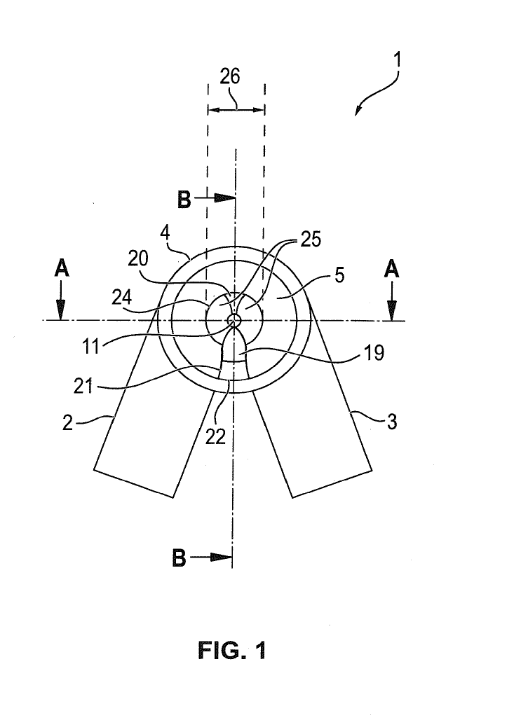

FIG. 1 schematically shows a bottom view of a device 1 according to the present invention for removing gas from a respiratory circuit of a respirator system. For clarity's sake, the further components of the respirator system are not shown. The further components are known to the person skilled in the art and are not the subject of the present invention. The respirator system has at least a respirator (also known as a ventilator) for setting and detecting respiration parameters or alternatively an anesthesia apparatus as well as an inhalation branch, which leads from the respirator to the device 1 and comprises at least one breathing tube, and an exhalation branch, which leads from the device to the respirator and comprises at least one breathing tube.

The device 1 has a first subsection (inhalation portion) 2, to which the inhalation branch of the respirator system, which inhalation branch is not shown, is connected. A second subsection (exhalation portion) 3 is connected to the exhalation branch of the respirator system, which exhalation branch is not shown. The first subsection 2 and the second subsection 3 open into a third subsection (patient adapter tube portion) 4, which has a tube connection opening 5, into which a tube adapter is inserted, so that a tube inserted into the trachea of a patient during the use of the device 1 can be inserted directly into the tube connection opening 5 via a tube adapter formed on the tube. The tube adapter is not shown in FIG. 1 for clarity's sake, and the tube as well as the intubated patient are not shown, either.

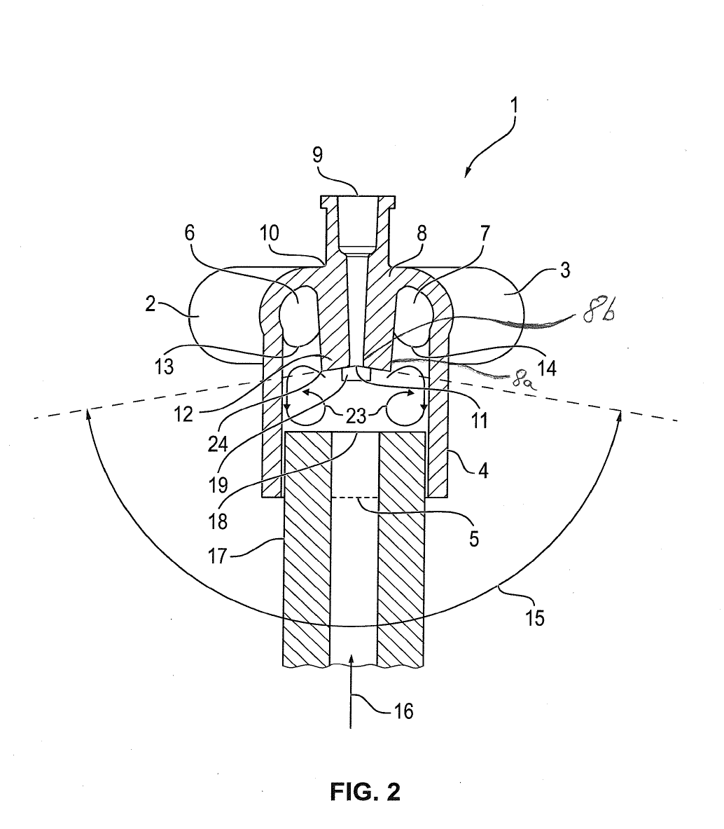

As can be schematically seen in FIG. 2, the first subsection 2 opens into the third subsection 4 with a first opening 6 and the second subsection 3 opens into the third subsection 4 with a second opening 7. The third subsection 4 extends in this exemplary embodiment at right angles to a plane defined by the first subsection 2 and the second subsection 3.

Both the first subsection 2 and the second subsection 3 may, of course, form an angle that is greater or preferably also smaller than 90° in relation to an axis extending through the third subsection 4.

Starting from an opening 9 in a wall 10 of the third subsection 4, a gas removal portion comprises a gas removal tube 8 that protrudes with a first end 12, having a removal opening 11, into the third subsection 4, so that the removal opening 11 is arranged essentially concentrically to the tube connection opening 5 and the first end 12 is located closer to the tube connection opening 5 than an edge 13 of the first opening 6 and than an edge 14 of the second opening 7. An end face 25 of the first end 12 connects an inner wall 8b of the gas removal tube 8, which inner wall surrounds the removal opening 11, with an edge 24 of the first end 12, which said end defines an outer wall 8a of the gas removal opening 8. As can be seen in FIG. 2, the end face 25 has a partially funnel-shaped design, i.e., it has a funnel-shaped area, which is interrupted by webs 19, 20, which will be described below. The funnel-shaped area of the end face 25 has an opening angle 15 between 130° and 170° and covers more than 40% of the end face available without webs. The funnel-shaped area of the end face 25 connects the edge 24 of the first end 12 with the inner wall 8b surrounding the removal opening 11, while the first end 12 tapers at the same time linearly in the direction of the exhaled breathing gas flow removed via the removal opening 11 from a diameter 26 at the edge 24 of the first end 12 to the diameter of the removal opening 11.

In one exemplary embodiment, not shown, the tapering may, of course, also take place by means of a bent curve opening towards the tube connection opening or a step likewise opening towards the tube connection opening.

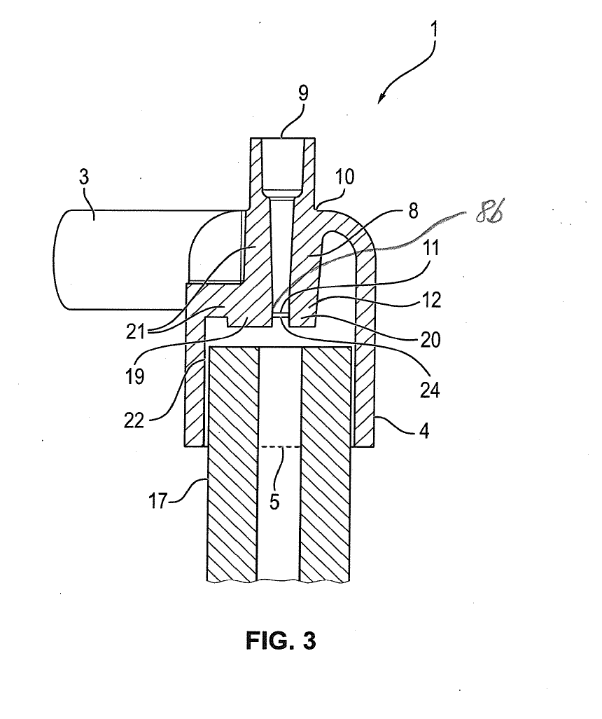

If, as is shown schematically in FIGS. 2 and 3, a tube adapter 17 is, for use, inserted into the third subsection 4, the funnel-shaped area of the end face 25 covers an opening 18 in the tube adapter in a screen-like manner. Exhaled breathing gas flows from the tube adapter 17 during a phase of exhalation predominantly in a direction 16 from the opening 18 into the third subsection 4. Due to the fact that the bisecting line of the opening angle 15 is parallel in the exemplary embodiment to the direction 16, the opening 18 is covered uniformly by the funnel-shaped area of the end face 25.

One part of the exhaled breathing gas flowing out of opening 18 flows partly directly into the removal opening 11 and another part flows toward the funnel-shaped area of the end face 25, so that exhaled breathing gas flowing towards the funnel-shaped area of the end face 25 is deflected partly back in the direction of the tube adapter 17, as a result of which swirls 23 are generated, forming a swirl barrier. The generated swirls reach, as is schematically shown in FIG. 2, into an area (a swirl barrier area) between the edge 13 of the first opening 6 as well as the edge 14 of the second opening 7 and the first end 12, extend in the direction of the tube adapter 17 along an inner wall of the third subsection 4 and flow back again together with the exhaled breathing gas flowing from the tube adapter 17 in the direction of the first end 12 or the funnel-shaped area of the end face 25. Inhaled breathing gas, which is introduced into the third subsection 4 from the first subsection 2 during the operation of the respirator system during the phase of exhalation, flows into the area in which the swirls 23 are formed at a lower concentration than this would happen without swirls. The gas mostly flows through a bypass region to the third subsection 3. In particular, the swirls 23 make it more difficult for inhaled breathing gas (gas introduced from the first subsection 2) to enter the area of the removal opening 11 from the first subsection 2 during the phase of exhalation. Exhaled breathing gas flowing from the opening 18 in the tube adapter 17 rather enters from the inhalation branch connected at the first subsection 2 into the removal opening 11 and from there via the opening 9 in the wall and a tube, not shown, and farther into an analyzing device, likewise not shown, in which, for example, the concentration of the carbon dioxide in the exhaled breathing gas is determined, without being mixed with the oxygen-containing inhalation branch.

Due to the special design of the first end 12 of the gas removal tube 8, with the end face 25 being funnel-shaped over at least 40% with an opening angle between 130° and 170°, inhaled breathing gas flowing from the first subsection 2 into the third subsection 4 during the phase of exhalation is carried on the swirls 23 like on an air cushion and predominantly deflected around the gas removal tube 8 and it then enters the exhalation branch connected at the second subsection 3 from the device 1 via the second opening 7 in the second subsection 3.

To impart a preferred direction to an inhaled breathing gas flow from the first subsection 2 to the second subsection 3 and thereby promote the gliding on the swirls 23, a partition 21 is arranged between the gas removal tube 8 and an inner wall 22 of the third section 4, which said inner wall is arranged between the first opening 6 and the second opening 7, so that inhaled breathing gas cannot flow directly along the inner wall 21 from the first opening 6 into the second opening 7, but it must first flow around the gas removal tube 8 or a smaller portion of it must flow through under the partition 21 in order to enter the second opening 7.

The partition 21 is made in one piece with the inner wall 22 and with the gas removal tube 8 for an especially advantageous manufacture of the device 1.

It is, of course, also possible to attach the partition 21 by bonding, locking or fitting in with other fastening means commonly known to the person skilled in the art between the gas removal tube 8 and the inner wall 22.

Web 19 and web 20 adjoin the first end 12 of the gas removal tube 8. The webs 19, 20 have, on the side facing the tube adapter 17, an area that is essentially at right angles to an axis extending through the removal opening 11. The webs 19, 20 divide the end face 25 of the first end 12 into two partial areas, which together form the funnel-shaped area of the end face 25, and two flat areas formed by the webs 19, 20. The webs 19, 20 protrude farther into the third subsection 4 than the first end 12, as a result of which the tube adapter 17 is caused to be able to be pushed in at the most to the extent that it is in contact with the web 19 and the web 20.

Exhaled breathing gas flowing from the opening 18 can thus flow towards the funnel-shaped area of the end face 25 even when the tube adapter 17 is in contact with the webs 19, 20, and it can be deflected from there laterally, so that the above-mentioned swirls 23 are formed.

It is, of course, also possible to provide only one web or more than two webs. However, two webs 19 and 20 are preferably used, because it is guaranteed hereby, on the one hand, that if the tube adapter 17 is pushed in too far, said tube adapter is in contact with two surfaces and is thus seated in the third subsection without being tilted. Tilting could occur in case of only one web if the tube adapter 17 has a clearance in the third subsection 4. On the other hand, the largest possible contiguous area of the end face of the first end 12 can be made funnel-shaped at the same time with a limitation to two webs.

It is especially favorable if the web 19 is connected in one piece with the partition 21 and with the first end 12, as is shown in the exemplary embodiment. Web 19, just as web 20, may, of course, also be fastened to the partition 21 and/or the end face of the first end 12 of the gas removal tube 8 by fastening possibilities known to the person skilled in the art, for example, bonding.

The bisecting line of the opening angle is parallel to the direction 16 in which exhaled breathing gas is discharged from the opening 18 of the tube adapter 17 in the exemplary embodiment shown in the figures. Swirling is achieved, of course, already when the bisecting line of the opening angle 15 is essentially parallel to the direction 16. “Essentially” is defined here such that a deviation from parallelism is on the order of magnitude of usual manufacturing tolerances, i.e., in the range of up to 1°. Swirls will, of course, also form if the bisecting line of the opening angle 15 is not essentially parallel to the direction 16 but has deviations of more than 1°. The general rule is that the closer the angle is to 0°, the more effective will be the formation of the swirls 23.

The gas removal tube 8 is arranged essentially concentrically to the tube connection opening 5 of the third subsection 4 in the exemplary embodiment. The gas removal tube 8 may, of course, also be introduced obliquely into the third subsection 4. It is only necessary that the removal opening 11 be arranged essentially concentrically to the tube connection opening 4 in order for the funnel-shaped area of the end face 25 to be able to cover the opening 18 usually arranged centrally in the tube adapter 17 uniformly in a screen-like manner.

The device 1 according to the present invention may be used both in a respirator system in intensive care and in an anesthesia system. The use of the device 11 according to the present invention in a respirator system that is used to respirate newborns is especially advantageous. Due to the tube adapter 17 being inserted directly into the device 1, the dead volume between the tube and the device 1 is kept as small as possible, while accurate measurement of the CO₂ content in the exhaled breathing gas is possible at the same time due to the design of the first end 12 of the gas removal tube 8 according to the present invention even when a flow of inhaled breathing gas is sent through the device 1 during the phase of exhalation.

While specific embodiments of the invention have been shown and described in detail to illustrate the application of the principles of the invention, it will be understood that the invention may be embodied otherwise without departing from such principles.

1. A device for removal of gas from a respiratory circuit, the device comprising:
a first subsection for connection to an inhalation branch of a respirator system;
a second subsection for connection to an exhalation branch of the respirator system;
a third subsection with a tube connection opening for insertion of a tube adapter, wherein the first subsection opens into the third subsection via a first opening and the second subsection opens into the third subsection via a second opening; and
a gas removal tube extending from an opening in a wall of the third subsection into the third subsection, the removal tube having a first end having a removal opening, so that when the tube adapter is inserted, the removal opening is arranged essentially concentrically to an opening formed in the tube adapter and the first end is arranged under an edge of the first opening and an edge of the second opening, in relation to a direction in which exhaled breathing gas is predominantly discharged from the opening, in the tube adapter when the device is used, wherein the first end has an end face, which connects an edge of the first end, which said edge defines an outer wall of the gas removal tube, with an inner wall of the gas removal tube, which said inner wall surrounds the removal opening, wherein at least 40% of the end face is made funnel-shaped with an opening angle between 130° and 170°.

2. A device in accordance with claim 1, wherein a bisecting line of the opening angle is essentially parallel to the direction in which exhaled breathing gas is predominantly discharged from the opening of the connected tube adapter into the third subsection during the use of the device.

3. A device in accordance with claim 1, wherein a ratio of a diameter of the first end at the edge to a diameter of the tube connection opening is between 0.35 and 0.65.

4. A device in accordance with claim 1, wherein a ratio of a diameter of the removal opening to a diameter of the first end at the edge is between 0.125 and 0.4.

5. A device in accordance with claim 1, further comprising at least one web, which extends farther into the third subsection than does the first end, the at least one web being connected to the first end of the gas removal tube, so that the tube adapter can be inserted into the third subsection at most up to the web.

6. A device in accordance with claim 5, further comprising a partition connecting a portion of the gas removal tube with an inner wall of the third subsection, which said inner wall is arranged between the first opening and the second opening, such that when the device is used, inhaled breathing gas flowing from the first opening towards one side of the partition must flow around the gas removal tube and/or must flow under the partition in order to be able to reach the other side of the partition and flow out the second opening.

7. A device in accordance with claim 6, wherein the web and the partition are made in one piece.

8. A device in accordance with claim 1, wherein the third subsection extends essentially at right angles to a plane defined by the first subsection and the second subsection.

9. (canceled)

10. A device in accordance with claim 1, further comprising a partition connecting a portion of the gas removal tube with an inner wall of the third subsection, which said inner wall is arranged between the first opening and the second opening, whereby during use, inhaled breathing gas flows from the first opening to the second opening at least one of around the gas removal tube and under the partition.

11. A respirator system comprising:
a respirator device or anesthesia device with an inhalation branch and an exhalation branch; and
a device connected to the inhalation branch and an exhalation branch, the device comprising:
a first subsection connected to an inhalation branch;
a second subsection connected to the exhalation branch;
a third subsection with a tube connection opening for insertion of a tube adapter, wherein the first subsection opens into the third subsection via a first opening and the second subsection opens into the third subsection via a second opening; and
a gas removal tube extending from an opening in a wall of the third subsection into the third subsection, the gas removal tube having a first end having a removal opening, so that when the tube adapter is inserted, the removal opening is arranged essentially concentrically to an opening formed in the tube adapter and the first end is arranged under an edge of the first opening and an edge of the second opening, in relation to a direction in which exhaled breathing gas is predominantly discharged from the opening, in the tube adapter when the device is used, wherein the first end has an end face, which connects an edge of the first end, which said edge defines an outer wall of the gas removal tube, with an inner wall of the gas removal tube, which said inner wall surrounds the removal opening, wherein at least 40% of the end face is made funnel-shaped with an opening angle between 130° and 170°.

12. A respirator system in accordance with claim 11, wherein a bisecting line of the opening angle is essentially parallel to the direction in which exhaled breathing gas is predominantly discharged from the opening of the connected tube adapter into the third subsection during the use of the device.

13. A respirator system in accordance with claim 11, wherein a ratio of a diameter of the first end at the edge to a diameter of the tube connection opening is between 0.35 and 0.65.

14. A respirator system in accordance with claim 11, wherein a ratio of a diameter of the removal opening to a diameter of the first end at the edge of the first end is between 0.125 and 0.4.

15. A respirator system in accordance with claim 11, further comprising at least one web extending a distance farther into the third subsection than an extent of the first end, the at least one web being connected to the first end of the gas removal tube, so that the tube adapter can be inserted into the third subsection at most up to the web.

16. A respirator system in accordance with claim 15, further comprising a partition connecting a portion of the gas removal tube with an inner wall of the third subsection, which said inner wall is arranged between the first opening and the second opening, such that when the device is used, inhaled breathing gas flowing from the first opening towards one side of the partition must flow around the gas removal tube and/or must flow under the partition in order to be able to reach the other side of the partition and flow out the second opening.

17. A respirator system in accordance with claim 16, wherein the web and the partition are made in one piece.

18. A respirator system in accordance with claim 11, wherein the third subsection extends essentially at right angles to a plane defined by the first subsection and the second subsection.

19. A respiratory inhalation branch and an exhalation branch to patient connection device comprising:
an inhalation portion with an inhalation branch connection interface;
an exhalation portion with an exhalation branch interface;
a patient adapter tube portion with an adapter tube connection interface with a tube connection for receiving a tube adapter, wherein the inhalation portion opens into the patient adapter tube portion via an inhalation portion opening and the exhalation portion opens into the patient adapter tube portion via a exhalation portion opening; and
a gas removal portion comprising a gas removal interface, a gas removal opening facing a tube adapter opening region of the adapter tube connection interface and providing a gas passage from the gas removal interface to the patient adapter tube portion, a swirl barrier region, a bypass region and a funnel shaped area, adjacent to and essentially concentric to the gas removal opening, for forming gas swirls in the swirl barrier region, wherein in a gas removal use state, with the tube adapter connected to the adapter tube connection interface, gas exhaled by the patient passes through the adapter tube and into the gas removal portion with a portion of the gas exhaled entering the gas removal opening, and a portion of the gas exhaled being directed towards the funnel shaped area and swirling in the swirl barrier region, to at least partially bar gas from flowing from the inhalation portion into the gas removal opening, whereby gas flowing from the inhalation portion predominantly flows to the exhalation portion via the bypass region.

20. A connection device according to claim 19, wherein the gas removal portion includes a gas removal tube extending from an opening in a wall of the patient adapter tube portion into the patient adapter tube portion, the tube having a first end with the gas removal opening, the tube having an end face with an outer edge, which defines an outer wall of the gas removal tube, and with an inner wall, which surrounds the removal opening, wherein at least 40% of the end face comprises the funnel shaped area and the funnel shaped area has an opening tapering angle between 130° and 170°, the tapering angle being formed by one of a linear or curved end face surface.
